(19) United States
(12) Patent Application Publication
(10) Pub. No.: US 2015/0196602 A1
(43) Pub. Date: Jul. 16, 2015

(54) STEM CELL THERAPY FOR CROHN'S DISEASE AND ULCERATIVE COLITIS

(71) Applicant: Burton FEINERMAN, TAMPA, FL (US)

(72) Inventor: Burton FEINERMAN, TAMPA, FL (US)

(21) Appl. No.: 14/597,010

(22) Filed: Jan. 14, 2015

Related U.S. Application Data
(60) Provisional application No. 61/927,128, filed on Jan. 14, 2014.

(51) Int. Cl. A61K 35/28

(52) U.S. Cl. CPC A61K 35/28

(57) ABSTRACT

A therapeutic process for treating an inflammatory bowel disease (IBD) using mesenchymal stem cells includes the steps of isolating a plurality of human mesenchymal stem cells from a human patient, introducing the plurality of human mesenchymal stem cells into the human patient, reducing human pro-inflammatory cytokines, wherein the human pro-inflammatory cytokines include IL-12 and IL-18, and increasing human anti-inflammatory cytokines, wherein the human anti-inflammatory cytokines include IL-10 and TGF-beta. The human mesenchymal stem cells are isolated from an umbilical cord blood or from a human adipose tissue. The plurality of human mesenchymal stem cells is introduced into the human patient through a intravenous administration or through an enema. The plurality of human mesenchymal stem cells is reconstituted into growth factor rich plasma prior to being introduced into the human patient.

The current application claims a priority to the U.S. Provisional Patent Application Ser. No. 61/927,128, filed on Jan. 14, 2014.

### FIELD OF THE INVENTION

The present invention relates generally to a method stem cell therapy. More specifically, it is a method of stem cell therapy for treatment of Crohn's disease and ulcerative colitis.

### BACKGROUND OF THE INVENTION

Crohn's disease and ulcerative colitis represent the two most common forms of inflammatory bowel disease (IBD). Crohn's disease (CD) may affect any part of the gastrointestinal tract, most commonly affecting the small bowel and the end of the colon. While ulcerative colitis (UC) affects only the colon.

Crohn's disease (CD) is a type of inflammatory bowel disease (IBD) that may affect any part of the gastrointestinal tract from mouth to anus. Symptoms often include abdominal pain, diarrhea (which may be bloody if inflammation is severe), fever and weight loss. Other complications may occur outside the gastrointestinal tract and include anemia, skin rashes, arthritis, inflammation of the eye, and tiredness. Bowel obstruction also commonly occurs and those with the disease are at greater risk of bowel cancer.

Ulcerative colitis (UC) is a form of inflammatory bowel disease. UC symptoms vary according to the location of the damage, such as ulcerative proctitis (recto-anal), proctosigmoiditis (sigmoid colon with bloody diarrhea and abdominal cramps), left sided colitis, pancolitis (entire colon) and fulminant colitis that can lead to rupture of the colon.

Ulcerative colitis has an incidence of 1 to 20 cases per 100,000 individuals per year, and a prevalence of 8 to 246 per 100,000 individuals.

Ulcerative colitis can be treated by performing a total colectomy. On the other hand, surgery for Crohn's disease involves removing the damaged parts of the intestine and reconnecting the healthy parts, which does not cure Crohn's, as it can recur after surgery, mostly at the site of the intestinal anastomosis (connection) or in other areas. Ulcerative colitis is an intermittent disease, with periods of exacerbated symptoms, and periods that are relatively symptom free. Although the symptoms of ulcerative colitis can sometimes diminish on their own, the disease usually requires treatment to go into remission. Treatments may be similar for both diseases such as Azulfidine, Asacol and the use of corticosteroids. Treatment using these medications of the mild forms may be helpful but of limited value with moderate and severe forms of the diseases.

In addition, both conditions are considered autoimmune diseases associated with chronic inflammation of the intestinal tract due to immune reactions from increased T lymphocyte activity and release of inflammatory cytokines. Autoimmune antibodies are released that target the lining of the intestinal tract. The release of leukocytes into the intestinal mucosa produces lesions from pro-inflammatory cytokines and inflammatory mediators. Defects in Regulatory T lymphocytes (Tregs) lead to damage seen in IBD. Activation of Thy1 and Thy2 helper cells sets off the immunologic cascade with activation of dendritic cells, cytokines, IFN-gamma, IL-12 and IL-18.

There is currently no cure for these disorders and current treatments do not work in a regular fashion from individual to individual and often have potential serious side effects. In light of the foregoing, it is one objective of the present invention is to provide a novel therapeutic approach that takes advantages of the respective patient's stem cell to treat the inflammatory bowel disease (IBD), including Crohn's disease (CD) and ulcerative colitis (UC).

### DETAIL DESCRIPTIONS OF THE INVENTION

All illustrations of the drawings are for the purpose of describing selected versions of the present invention and are not intended to limit the scope of the present invention.

The present invention is a therapeutic approach with stem cell and specific anti-inflammatory factors to treat inflammatory bowel disease (IBD), including Crohn's disease (CD) and ulcerative colitis (UC). The therapeutic approach of the present invention targets the specific causes of inflammatory bowel disease.

The initial procedure of this therapeutic approach is to isolate human mesenchymal stem cells (MSC). The human MSC is isolated from human adipose tissue or other origins following the process set forth below.

Draw 60 ml of blood at the antecubital space from a patient. Centrifuge the blood for 10 minutes at 100×g at room temperature of 72 degree F. Aspirate the supernatant obtained following the foregoing centrifugation and aliquot into 15 ml centrifuge tubes 5 ml sample volume in each of 2 tubes. Subsequently centrifuge for 10 minutes at 30×g at room temperature of 72 degree F., p), and then remove most of the supernatant and leave only 1.5 ml in each tube. Reconstitute pellet in both tubes and add contents of one tube to the other. Add 1 mg of a calcium channel activator. Allow the sample to clot and then add 60 ml growth factor rich plasma. In addition, perform a mini-liposuction from lower abdomen of the patient. Wash fat 100 gram with saline several times. Add cell extraction medium. Centrifuge at 1000×g for 5 minutes. Discard supernatant and obtain a pellet. Reconstitute the pellet in centrifuge tube using 8 ml fresh saline and then centrifuge at 1000×g for 5 minutes. Last, reconstitute the obtained pellet that has 25 million cells by mixing with the previous obtained Growth factor Rich Plasma 60 ml.

Subsequently, inject intravenously the abovementioned reconstituted human MSC (pellet plus Growth factor Rich Plasma) 60 ml into the patient.

Further, it is known that intestinal epithelium is constantly shedding and replaced by rapidly dividing stem cells which are a target for stem cell therapy. AAV vectors are able to perform transduction of intestinal stem cells with long term integration and expression. Intravenous administration of AAV vectors also has been shown to have long term transduction of the intestinal epithelium in both Crohn's disease (CD) and ulcerative colitis (UC). In the present invention, the molecular targets of both diseases are the proinflammatory cytokines IL-12 and IL-18. Moreover, in the present invention, two anti-inflammatory cytokines IL-10 and TGF-beta have been used or enhanced.

AAV vectors allow stable long term transduction of MSC in Crohn's disease as well as transport effectively into the intestinal epithelium agents to target pro-inflammatory cytokines and over-expression of anti-inflammatory cytokines for both CD and UC.

Mice with chronic T cell mediated colitis (trinitrobenzene sulfonic acid induced colitis in mice) were treated over an eight day period in which mesenchymal stem cells (MSC) isolated from umbilical cord blood transfected with AAV-10 vectors and introduced by enema 90 ml into the lumen of the colon every other day and also intraperitoneally one time. On the tenth day the mice were given anti-inflammatory cytokines IL-10 and TGF-beta gene fragments constructed with AAV-10 and is then expressed as proteins into the colon lumen and intraperitoneally.

In addition, the same treatment was done every day over a time period of eight days. The mice with chronic T cell mediated Crohns disease were given mesenchymal stem cells constructed with AAV-10 vectors administered through a feeding tube into the duodenum. On the tenth day AAV-10 vectors with IL-10 and TGF-beta genes and given by feeding tube into the duodenum.

In both cases the mice were noted to have reduced levels of IL-12 and IL-18 and increased levels of IL-10 and TGF-beta. The MSC that were used in these experiments were obtained from human umbilical cord blood.

In one working example of the present invention in human, following the injection of human MSC, a patent demonstrates the beneficial effects of immune- modulatory function and ability to differentiate into intestinal cells.

Five human patients with CD and five patients with UC were treated based on the above treatments in laboratory animals. The five patients with CD ages 19, 21, 24, 29, 30 had similar symptoms with bloody diarrhea 2-5 times a day, weight loss, abdominal cramps on a daily basis prior to the treatment. The treatment consisted on construction of AAV-10 vector (has DNA insertion) with mesenchymal stem cells 50 million isolated from lower abdominal adipose tissue on Day 1 given intraperitoneally and by duodenal tube on Day 2. Two days later the patients received AAV-10 vectors constructed with mesenchymal stem cells 50 million plus IL-10 and TGF-beta. The patients were seen at 3 months, 6 months, and 12 months. They had each had gained weight; had only occasional loose stools without blood and no further abdominal cramps.

Five patients ages 23, 24, 27, 32 36 with UC had 3-4 loose to watery stools daily with blood or mucus; abdominal pain; tenesmus; low grade temperatures 99-100.2 F; tenesmus. Their treatment consisted of AAV-10 vectors constructed with mesenchymal stem cells extracted from lower abdominal adipose tissue 100 million MSC given intravenously and by enema given on two occasions 24 hours apart. Two days after that the patients received AAV-10 vectors constructed with 200 i.u. IL-10 and TGF-beta given intravenously. The patients were followed at 3, 6 and 12 months. The patients showed similar improvement with only occasionally a soft stool but no further diarrhea with blood or mucus; no further abdominal pain; no tenesmus; no further low grade temperatures.

Although the invention has been explained in relation to its preferred embodiment, it is to be understood that many other possible modifications and variations can be made without departing from the spirit and scope of the invention as herein described.

What is claimed is:

1. A therapeutic process for treating an inflammatory bowel disease (IBD) using mesenchymal stem cells, comprising the steps of
isolating a plurality of human mesenchymal stem cells from a human patient; and
introducing the plurality of human mesenchymal stem cells into the human patient.

2. The therapeutic process for treating an inflammatory bowel disease (IBD) using mesenchymal stem cells as claimed in claim 1, wherein the inflammatory bowel disease comprising
Crohn's disease (CD); and
ulcerative colitis (UC).

3. The therapeutic process for treating an inflammatory bowel disease (IBD) using mesenchymal stem cells as claimed in claim 1, wherein the inflammatory bowel disease comprising
the human mesenchymal stem cells being isolated from an umbilical cord blood.

4. The therapeutic process for treating an inflammatory bowel disease (IBD) using mesenchymal stem cells as claimed in claim 1, comprising
the human mesenchymal stem cells being isolated from a human adipose tissue.

5. The therapeutic process for treating an inflammatory bowel disease (IBD) using mesenchymal stem cells as claimed in claim 1, comprising
the plurality of human mesenchymal stem cells being introduced into the human patient through a intravenous administration.

6. The therapeutic process for treating an inflammatory bowel disease (IBD) using mesenchymal stem cells as claimed in claim 1, comprising
the plurality of human mesenchymal stem cells being introduced into the human patient through an enema.

7. The therapeutic process for treating an inflammatory bowel disease (IBD) using mesenchymal stem cells as claimed in claim 1, comprising
the plurality of human mesenchymal stem cells being reconstituted into a growth factor rich plasma prior to being introduced into the human patient.

8. The therapeutic process for treating an inflammatory bowel disease (IBD) using mesenchymal stem cells as claimed in claim 7, wherein the growth factor rich plasma is obtained through the steps set forth below:
drawing 60 ml of blood from the human patient;
centrifuging at 100×g for 10 minutes to obtain a first supernatant;
aspirating and aliquoting the first supernatant into a plurality of 15 ml centrifuge tubes;
centrifuging at 30×g for 10 minutes to obtain a second supernatant and a first pellet in each of the plurality of 15 ml centrifuge tubes;
removing the second supernatant to allow only 1.5 ml of the second supernatant remaining in each of the plurality of 15 ml centrifuge tubes;
reconstituting the 1.5 ml second supernatant and the first pellet to obtain a first suspension in each of the plurality of 15 ml centrifuge tubes;
combining the first suspension in each of the plurality of 15 ml centrifuge tubes to obtain a total second suspension;
adding a pre-defined amount of calcium activator into the total second suspension; and
allowing the total second suspension to clot and to obtain the growth factor rich plasma.

9. The therapeutic process for treating an inflammatory bowel disease (IBD) using mesenchymal stem cells as claimed in claim 8, wherein plurality of human mesenchymal stem cells are obtained through the steps set forth below:
performing a mini-liposuction from a lower abdomen of the patient to obtain a predefined amount of fat;
washing the fat with a normal saline for a plurality of times;
adding a predefined amount of cell extraction medium;
centrifuging at 1000×g for a first pre-defined time period to obtain a third supernatant and a second pellet;
discarding the third supernatant;
adding 8 ml of normal saline;
reconstituting the second pellet;
centrifuging at 1000×g for a second pre-defined time period to obtain a fourth supernatant and a third pellet; and
discarding the fourth supernatant to obtain the plurality of human mesenchymal stem cells.

10. The therapeutic process for treating an inflammatory bowel disease (IBD) using mesenchymal stem cells as claimed in claim 1, further comprising
reducing human pro-inflammatory cytokines, wherein the human pro-inflammatory cytokines comprising IL-12 and IL-18.

11. The therapeutic process for treating an inflammatory bowel disease (IBD) using mesenchymal stem cells as claimed in claim 1, further comprising
increasing human anti-inflammatory cytokines, wherein the human anti-inflammatory cytokines comprising IL-10 and TGF-beta.

12. A therapeutic process for treating an inflammatory bowel disease (IBD) using mesenchymal stem cells, comprising the steps of
isolating a plurality of human mesenchymal stem cells from a human patient; and
introducing the plurality of human mesenchymal stem cells into the human patient;
wherein the human mesenchymal stem cells is isolated from an umbilical cord blood or from a human adipose tissue, and the plurality of human mesenchymal stem cells is introduced into the human patient through a intravenous administration or through an enema.

13. The therapeutic process for treating an inflammatory bowel disease (IBD) using mesenchymal stem cells as claimed in claim 12, wherein the inflammatory bowel disease comprising
Crohn's disease (CD); and
ulcerative colitis (UC).

14. The therapeutic process for treating an inflammatory bowel disease (IBD) using mesenchymal stem cells as claimed in claim 12, comprising
the plurality of human mesenchymal stem cells being reconstituted into a growth factor rich plasma prior to being introduced into the human patient.

15. The therapeutic process for treating an inflammatory bowel disease (IBD) using mesenchymal stem cells as claimed in claim 14, wherein the growth factor rich plasma is obtained through the steps set forth below:
drawing 60 ml of blood from the human patient;
centrifuging at 100×g for 10 minutes to obtain a first supernatant;
aspirating and aliquoting the first supernatant into a plurality of 15 ml centrifuge tubes;
centrifuging at 30×g for 10 minutes to obtain a second supernatant and a first pellet in each of the plurality of 15 ml centrifuge tubes;
removing the second supernatant to allow only 1.5 ml of the second supernatant remaining in each of the plurality of 15 ml centrifuge tubes;
reconstituting the 1.5 ml second supernatant and the first pellet to obtain a first suspension in each of the plurality of 15 ml centrifuge tubes;
combining the first suspension in each of the plurality of 15 ml centrifuge tubes to obtain a total second suspension;
adding a pre-defined amount of calcium activator into the total second suspension; and
allowing the total second suspension to clot and to obtain the growth factor rich plasma.

16. The therapeutic process for treating an inflammatory bowel disease (IBD) using mesenchymal stem cells as claimed in claim 15, wherein plurality of human mesenchymal stem cells are obtained through the steps set forth below:
performing a mini-liposuction from a lower abdomen of the patient to obtain a predefined amount of fat;
washing the fat with a normal saline for a plurality of times;
adding a predefined amount of cell extraction medium;
centrifuging at 1000×g for a first pre-defined time period to obtain a third supernatant and a second pellet;
discarding the third supernatant;
adding 8 ml of normal saline;
reconstituting the second pellet;
centrifuging at 1000×g for a second pre-defined time period to obtain a fourth supernatant and a third pellet; and
discarding the fourth supernatant to obtain the plurality of human mesenchymal stem cells.

17. The therapeutic process for treating an inflammatory bowel disease (IBD) using mesenchymal stem cells as claimed in claim 12, further comprising
reducing human pro-inflammatory cytokines, wherein the human pro-inflammatory cytokines comprising IL-12 and IL-18; and
increasing human anti-inflammatory cytokines, wherein the human anti-inflammatory cytokines comprising IL-10 and TGF-beta.

18. A therapeutic process for treating an inflammatory bowel disease (IBD) using mesenchymal stem cells, comprising the steps of
isolating a plurality of human mesenchymal stem cells from a human patient;
introducing the plurality of human mesenchymal stem cells into the human patient;
reducing human pro-inflammatory cytokines, wherein the human pro-inflammatory cytokines comprising IL-12 and IL-18; and
increasing human anti-inflammatory cytokines, wherein the human anti-inflammatory cytokines comprising IL-10 and TGF-beta,
wherein the human mesenchymal stem cells is isolated from an umbilical cord blood or from a human adipose tissue, the plurality of human mesenchymal stem cells is introduced into the human patient through a intravenous administration or through an enema, the plurality of human mesenchymal stem cells is reconstituted into a growth factor rich plasma prior to being introduced into the human patient, and the inflammatory bowel disease comprises Crohn's disease (CD) and ulcerative colitis (UC).

19. The therapeutic process for treating an inflammatory bowel disease (IBD) using mesenchymal stem cells as claimed in claim 18, wherein the growth factor rich plasma is obtained through the steps set forth below:
drawing 60 ml of blood from the human patient;
centrifuging at 100×g for 10 minutes to obtain a first supernatant;
aspirating and aliquoting the first supernatant into a plurality of 15 ml centrifuge tubes;
centrifuging at 30×g for 10 minutes to obtain a second supernatant and a first pellet in each of the plurality of 15 ml centrifuge tubes;
removing the second supernatant to allow only 1.5 ml of the second supernatant remaining in each of the plurality of 15 ml centrifuge tubes;
reconstituting the 1.5 ml second supernatant and the first pellet to obtain a first suspension in each of the plurality of 15 ml centrifuge tubes;
combining the first suspension in each of the plurality of 15 ml centrifuge tubes to obtain a total second suspension;
adding a pre-defined amount of calcium activator into the total second suspension; and
allowing the total second suspension to clot and to obtain the growth factor rich plasma.

20. The therapeutic process for treating an inflammatory bowel disease (IBD) using mesenchymal stem cells as claimed in claim 19, wherein plurality of human mesenchymal stem cells are obtained through the steps set forth below:
performing a mini-liposuction from a lower abdomen of the patient to obtain a predefined amount of fat;
washing the fat with a normal saline for a plurality of times;
adding a predefined amount of cell extraction medium;
centrifuging at 1000×g for a first pre-defined time period to obtain a third supernatant and a second pellet;
discarding the third supernatant;
adding 8 ml of normal saline;
reconstituting the second pellet;
centrifuging at 1000×g for a second pre-defined time period to obtain a fourth supernatant and a third pellet; and
discarding the fourth supernatant to obtain the plurality of human mesenchymal stem cells.
